(19) United States
(12) Patent Application Publication
(10) Pub. No.: US 2002/0090406 A1
(43) Pub. Date: Jul. 11, 2002

(54) Tahitian noni juice on COX-1 and COX-2 and tahitian noni juice as a selective COX-2 inhibitor

(76) Inventors: Chen Xing Su, West Jordan, UT (US); Claude Jarakae Jensen, Cedar Hills, UT (US); Stephen Paul Story, Alpine, UT (US)

Correspondence Address: KIRTON & McCONKIE, 1800 Eagle Gate Tower, 60 East South Temple Street, Salt Lake City, UT, 84111, US

(21) Appl. No.: 10/006,014

(22) Filed: Dec. 4, 2001

Related U.S. Application Data
(60) Provisional application No. 60/251,416, filed on Dec. 5, 2000.

(51) Int. Cl. A61K 35/78

(52) U.S. Cl. 424/765; 424/769

(57) ABSTRACT

A method for treating people with various ailments and diseases by administering an effective amount of processed Morinda citrifolia.

### RELATED APPLICATIONS

This application claims priority to U.S. Provisional Application No. 60/251,416 to Chen Xing Su et al filed Dec. 5, 2000 entitled COX-1 and COX-2 Inhibition Study on TNJ.

### BACKGROUND

1. Field of the Invention

This invention relates to a method and composition for treating inflammation and related painful conditions, more particularly, to administering processed Morinda citrifolia for the purpose of relieving pain and inflammation.

2. Background

A popular treatment for chronic pain and inflammation involves the use of nonsteroidal anti-inflammatory drugs (NSAIDs). NSAIDS are particularly useful in treating joint pain, muscle pain, and joint swelling. There are many different types of NSAIDs, including aspirin and other salicylates. Examples include ibuprofen, (e.g., Advil®, Motrin®, Nuprin®) naproxen, sulindac, diclofenac, piroxicam, ketoprofen, diflunisal, nabumetone, etodolac, oxaprozin, and indomethacin. Popular NSAIDs include: ibuprofen, naproxen and aspirin.

While NSAIDs have been effective in reducing inflammation and pain associated with it, NSAIDs have a number of adverse side effects. The major side effects of NSAIDs are gastrointestinal related. For example, between 10 and 50 percent of the patients being treated with NSAIDs suffer side effects such as diarrhea, heartburn, increased abdominal pain, and upset stomach. A significant percentage of these patients also develop ulcers in the stomach and upper GI tract, which can lead to internal bleeding and other complications.

Since significant numbers of patients taking NSAIDs were suffering from an increased risk of ulceration in the stomach, researchers began investigating the mechanisms by which NSAIDs inhibit and prevent inflammation. Researchers knew that in most instances, inflammation in human tissues (and the pain associated with it) is related to the conversion of arachidonic acid (a molecule present in the majority of human body cells) into a prostaglandin in the cells of the tissue. The conversion arachidonic acid to a prostaglandin requires the presence of an enzyme known as cyclooxygenase (COX). NSAIDS were known to inhibit the COX enzyme and thereby prevent or reduce inflammation.

As researchers studied the COX inhibitory activity of NSAIDs, they discovered that there are in fact two different COX enzymes: COX-1 and COX-2. COX-1 and COX-2 are isoform of cyclooxygenase and both of which catalyze the first two steps in the biosynthesis from arachidonic acid to the prostaglandins. The difference is that COX-1 is constitutive and COX-2 is inducible. COX-1 presents in nearly all parts of body at a constant level to produce the prostaglandins to line the stomach, maintain normal renal function, prevent platelet aggregation. On the other hand, COX-2 is normally absent from body and induced at the infected sites by those associated with inflammation such as bacterial polysaccharide and cytokines, interleukin-1, -2, and tumor necrosis factor. Once induced, COX-2 produces large amount of prostaglandins which lower the pain threshold (causes pain), raise the set point of the temperature-regulating center (causes fever), cause peripheral vasodilatation with local redness and edema formation. Therefore, the inhibition of COX-1 will lead to a series of side effects such as gastrointestinal ulceration and bleeding, renal damage, and platelet dysfunction, while the selective inhibition of COX-2 offers advantage of inhibition of inflammatory without disturbing nonnal body functions.

Researcher discovered that most “first generation” NSAIDs inhibit the enzymatic activities of both COX-1 and COX-2, and do not selectively inhibit COX-2 enzyme. Therefore, when a patient takes a typical NSAIDs, COX-2 is inhibited and inflammation is thereby reduced, but COX-1 is also inhibited.

In order to provide relief from inflammation associated with COX-2 without losing the COX 1 enzyme, drug companies have attempted to produce drugs that selectively inhibit COX2 without inhibiting COX-1. Selective COX-2 inhibition drugs have been developed and made available to the public for several years now. These selective COX-2 inhibition drugs were initially thought to be of special benefit to arthritis sufferers and those with chronic pain due to inflammation.

Even though selective COX-2 inhibition drugs have been reported to be a “success,” there are doubts about manufacturers' claims that selective COX-2 inhibition drugs are “safer” than non-selective COX inhibitors. Some of the side effects associated with non-selective COX inhibitors are also found to be associated with selective COX-2 inhibition drugs. More importantly, people using selective COX-2 inhibition drugs have been shown to have four times the risk of suffering a heart attack than those taking traditional, non-selective NSAIDs. By not inhibiting the COX-1 enzyme, selective COX-2 inhibition drugs were intended to be safer than the non-selective NSAIDs. However, there appears to be considerable risk associated with prolonged use of selective COX-2 inhibition drugs. At present, it is not known if the cause of the increased risk of heart attack associated with COX-2 inhibition is directly related to the inhibiting properties of the drug or if the increased risk of heart attack is the result of some other interaction with these particular selective COX-2 inhibition drugs. Ironically, some patients taking selective COX-2 inhibition drugs who are concerned with increased risk of heart attacks are attempting to reduce the risk by taking aspirin and other traditional non-selective NSAIDs along with the selective COX-2 inhibition drugs.

Other problems associated with the selective COX-2 inhibition drugs further complicate the ability of healthcare providers to easily and effectively treat patients suffering from inflammation. For example, in most cases selective COX-2 inhibition drugs are available by prescription only. Thus, in order to obtain these drugs, the patients are required to visit the doctor and receive a diagnosis that calls for these prescription drugs. After the visit, the patient must, of course, obtain the drugs from the pharmacy with the associated inconvenience that this process entails. Obtaining prescription drugs is much more complicated than buying over the counter pharmaceuticals or remedies and the cost of the drugs is significant.

Another disadvantage associated with selective COX-2 inhibition drugs is that they are, at present, not approved for pediatric use. Selective COX-2 inhibition drugs are unavailable to children who unfortunately may be more distressed than an adult would be by the unpleasant side effects associated with non-selective NSAIDs. Approval of pediatric selective COX-2 inhibition drugs may take several years, if such drugs are approved at all.

Other disadvantages of selective COX-2 inhibition drugs presently available also include the dangers of uncertain drug interaction for patients who are taking other medications in addition to selective COX-2 inhibition drugs. Also, pregnant women cannot take the selective COX-2 inhibition drugs during certain periods of fetal development. It has been determined that selective COX-2 inhibition drugs have teratogenic effects on fetuses. Additionally, potential harm could come to the patient if a COX 2 selective inhibitor is taken at a time when the patient is not properly hydrated.

It would be advantageous to provide a formulation for the treatment of inflammation that reduces the gastrointestinal discomfort and other side effects associated with many present anti-inflammatory pharmaceuticals. It would also be advantageous to provide a method and formulation that reduces inflammation and the pain associated with inflammation and at the same time limits the adverse side effects, such as those associated with selective COX-2 inhibition drugs of the prior art.

### SUMMARY OF THE INVENTION

The present invention is directed toward a formulation and method for reducing and limiting inflammation and the pain associated with inflammation. One embodiment of the present invention uses a selective COX-2 inhibitor as an anti-inflammatory agent where use of the selective COX-2 inhibitor does not result in the unpleasant side effects associated with NSAIDs and selective COX-2 inhibition drugs presently available.

The present invention provides a method of treating various diseases and ailments, which comprises administering to a mammal a therapeutically effective amount of processed Morinda citrifolia. Morinda citrifolia is generally administered in the form of a juice, oil, capsule or as an ingredient in another food product. An advantage of using processed Morinda citrifolia is that treatment may be carried out without causing gastric side effects that can occur by using NSAIDs for prolonged periods.

In a preferred embodiment, the formulation comprises processed Morinda citrifolia juice, which has been discovered to have selective COX-2 inhibitor characteristics. The precise mechanism by which processed Morinda citrifolia selectively inhibits COX-2 is not known. A preferred method of the present invention comprises the consumption of processed Morinda citrifolia juice in therapeutic amounts.

### DETAILED DESCRIPTION OF THE INVENTION

It will be readily understood that the components of the present invention, as generally described herein, could be arranged and designed in a wide variety of different methods, configurations or formulations. Thus, the following more detailed description of the embodiments of the system and method of the present invention, is not intended to limit the scope of the invention, as claimed, but is merely representative of the presently preferred embodiments of the invention.

The Indian Mulberry plant, known scientifically as Morinda citrifolia L., is a shrub, or small or medium sized tree 3 to 10 meters high. It grows in tropical coastal regions around the world. The plant grows in the wild, and it has been cultivated in plantations and small individual growing plots. The Indian mulberry plant has somewhat rounded branches and evergreen, opposite (or spuriously alternate), dark, glossy, wavy, prominently-veined leaves. The leaves are broadly elliptic to oblong, pointed at both ends, 10-30 cm in length and 5-15 cm wide.

The Indian mulberry flowers are small, white, 3 to 5 lobed, tubular, fragrant, and about 1.25 cm long. The flowers develop into compound fruits composed of many small drupes fused into an ovoid, ellipsoid or roundish, lumpy body, 5-10 cm long, 5-7 cm thick, with waxy, white or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged, 2-celled stones, each containing about 4 seeds.

When fully ripe, the fruit has a pronounced odor like rancid cheese. Although the fruit has been eaten by several nationalities as food, the most common use of the Indian mulberry plant was as a red and yellow dye source. Recently, there has been an interest in the nutritional and health benefits of the Indian mulberry plant.

Because the Morinda citrifolia fruit is for all practical purposes inedible, the fruit must be processed in order to make it palatable for human consumption and included in food products used to treat various ailments and diseases. Processed Morinda citrifolia juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other processes include freeze drying the fruit and juice. The fruit and juice can be reconstituted during production of the final juice product. Still other processes include air drying the fruit and juices, prior to being masticated.

In a currently preferred process of producing Morinda citrifolia juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp. The juice can be filtered from the pulp.

The juice can be packaged into containers for storage and transport. Alternatively, the juice can be immediately processed into finished juice product. The containers can be stored in refrigerated, frozen, or room temperature conditions. The pulp can be blended in with the juice to make a puree. The Morinda citrifolia juice and puree can then be blended in a homogenous blend and mixed with other ingredients. The other ingredients consist of, but are not limited to water, fruit juice concentrates, flavorings, sweeteners, nutritional ingredients, botanicals, and colorings. The finished juice product is preferably heated and pasteurized at a minimum temperature of 181° F. (83° C.) or higher up to 212° F. (100° C.).

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

Pure juice can be processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration, or any other standard commercial filtration devices. The operating filter pressure preferably ranges from 0.1 psig up to about 1000 psig. The flow rate preferably ranges from 0.1 gpm up to 1000 gpm, and more preferably between 5 and 50 gpm.

In addition to the processing methods described above, other methods of processing fruit into oil product, fiber product, and juice product are contemplated and may be employed. Several embodiments of formulations of processed juice, oil, and fiber can be used.

Some embodiments of the present invention encompass a method of treating various diseases and ailments in a human which comprises administering to a mamal an effective amount of processed Morinda citrifolia.

The invention anticipates using processed Morinda citrifolia for the treatment of pain and inflammation, arthritis, dysmenorrhea, low back and neck pain and muscle strains and sprains.

The processed Morinda citrifolia may be modified to increase the benefits for particular diseases and ailments. Oral administration is a preferred mode of administration. In some embodiments, the invention encompasses pharmaceutical compositions in combination with processed Morinda citrifolia for inhibiting the production of the prostaglandins by COX-2) and treating the above-mentioned diseases and ailments comprising a pharmaceutically acceptable carrier, and a therapeutically effective amount of processed Morinda citrifolia described above. These could take the form of a tablet or capsule, solutions, or be included as an ingredient in another food product.

As with “pure” processed Morinda citrifolia, the compound may be useful for the relief of fever and inflammation of joints, low back and neck pain, dysmenorrhea, sprains and strains, and arthritis, including rheumatoid arthritis and degenerative joint diseases (osteoarthritis).

While the exact mechanisms by which processed Morinda citrifolia works are unknown, it is possible that Morinda citrifolia compounds thereof function in a manner similar to other selective COX-2 inhibitors and are thereby useful in the treatment of a variety of prostaglandin mediated diseases. This possibility is illustrated by Morinda citrifolia 's ability to selectively inhibit COX-(2).

The pharmaceutical compositions containing the active ingredient may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents. Tablets contain the active ingredient in admixture with non-toxic pharmaceutically acceptable excipients which are suitable for the manufacture of tablets. Aqueous suspensions contain the active material in admixture with excipients suitable for the manufacture of aqueous suspensions.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

It is a great advantage of this invention that treatment may be carried out without causing gastric side effects of the type that can occur when NSAIDs are used for prolonged periods. Thus, the provision of medicaments which are surprisingly effective without any significant tendency to cause gastric side effects at the therapeutic dose is of great use particularly to the elderly.

Favorably, this invention provides a method of treating inflammation and pain associated with it. This method comprises the oral administration of a pharmaceutical composition that inhibits COX-2, which causes pain and inflammation, while inhibiting to a lesser extent, COX-1, which keeps the normal functions of the body.

Generally, the oral dosage will be administered from two to three times per day.

### EXAMPLE 1

In an actual example, Morinda citrifolia juice was tested for COX-1 and COX-2 inhibition. Enzyme assays were conducted on COX-1 and COX-2. The source of the COX-1 enzymes was human platelet. The substrate was 50 million cell arachidonic acid in a 1% DMSO vehicle. Pre-incubation time for the COX-1 immuno assay was 15 minutes at 37° C., the incubation time and temperature was also 15 minutes at 37° C. An incubation buffer was HBSS buffer with 15 mMHEPS, at a pH of 7.4. EIA quantitation of the prostaglandin E2 was performed. A significance criteria of greater or equal to 50 percent of maximum stimulation or inhibition was employed.

With respect to the COX-2 enzyme assay, the source of the COX-2 was human recombinant Sf9 insect cells and the substrate wass 0.3 μm of arachidonic acid. The vehicle was a 1% DMSO. Preincubation time and temperature were 15 minutes at 37° C. Incubation time and temperature were 5 minutes at 37° C. The incubation buffer was 100 mM Tris-HCl, 1 mM glutathione, 1 uM hematin, and 500 uM of phenol at a pH of 7.7. EIA quantitation of the prostaglandin E₂ was performed. The significance criteria of greater than or equal to 50 percent of the maximum stimulation or inhibition was employed. The biochemical assay results show that at a concentration of 2.31 percent, inhibition of the COX-1 was 20 percent, while inhibition of the COX-2 was almost 60 percent. Where the concentration was increased to 10 percent, the inhibition of COX-1 is shown to be approximately 83 percent and the inhibition of COX-2 is approximately 84 percent. Thus, at greater concentrations, the selectivity of COX-2 or COX-1 seems to be limited. The results of this study indicate that at a given concentration of Morinda citrifolia juice, the inhibition of COX-2 was 58 percent and the inhibition of COX-1 was 20 percent. Morinda citrifolia juice shows selective COX-2 inhibition.

In addition to showing the selectivity for COX-2 inhibition of processed Morinda citrifolia juice, the study also suggests that COX-2 selectivity with Morinda citrifolia juice is sensitive or related to concentration. The study shows that different concnetrations produced different levels of selectivity between the enzymes. Because the mechanism by which Morinda citrifolia juice selectively inhibits COX-2 is not known, the reason for the difference in concentration results cannot be determined definitively based on these data. However, it is clear that where an excessive concentration of Morinda citrifolia juice is used, COX-2 selectivity is reduced. The COX-2 selectivity in a sense is undermined by excessive, increased concentration. An increased concentration of Morinda citrifolia juice may result in non-selective inhibition of both COX-1 and COX-2. These results suggest that limiting undesirable COX-1 inhibition by Morinda citrifolia juice may be accomplished by appropriately limiting the concentration. Thus, with respect to at least one embodiment of the present invention, the data suggest the surprising result that in some circumstances “less” Morinda citrifolia juice provides “more” inhibition selectivity.

### EXAMPLE 2

In this example, a patient is experiencing pain and inflammation. The individual desires to treat the condition with a nonprescription, over-the-counter preparation. To treat the infection, the individual consumes a predetermined amount of food product containing processed Morinda citrifolia. The person intermittently consumes the food product containing the processed Morinda citrifolia until the pain and inflammation is reduced or eliminated.

### EXAMPLE 3

In this example, a person is suffering from arthritis. To treat the pain associated with arthritis, the person consumes a prescribed amount of food product containing processed Morinda citrifolia. The person intermittently consumes the food product containing the processed Morinda citrifolia until the arthritic symptoms decrease or disappear.

### EXAMPLE 4

In this example, a person believes he or she is susceptible to a condition that results in chronic inflammation. In order to reduce the likelihood developing chronic inflammation, this person regularly consumes processed Morinda citrifolia juice in therapeutic doses.

### EXAMPLE 5

In this example, a person has developed a condition that results in chronic inflammation. To inhibit or reduce chronic inflammation, this person intermittently consumes processed Morinda citrifolia juice in therapeutic doses.

### EXAMPLE 6

In this example, a woman is suffering from menstrual pain related to inflammation. To decrease or eliminate this pain and to otherwise treat the cause of the menstrual pain related to inflammation, the woman intermittently consumes processed Morinda citrifolia juice in therapeutic doses.

### EXAMPLE 7

In this example, a person is suffering from low back and neck pain. To treat this pain, this person regularly consumes food products containing processed Morinda citrifolia.

### EXAMPLE 8

In this example, a person is suffering from muscle strains and sprains. To decrease or eliminate the pain associated with such strains and sprains and to help in the healing process, this person intermittently consumes food products containing processed Morinda citrifolia.

What is claimed is:

1. A method of treating pain and inflammation comprising the steps of:
providing a dose of Morinda citrifolia for consumption and limiting undesired COX-1 inhibition relative to COX-2 inhibition by limiting the concentration of said dose.

2. The method of claim 1, wherein the Morinda citrifolia is processed.

3. The method of claim 1, wherein the Morinda citrifolia is in liquid form.

4. The method of claim 1, wherein the Morinda citrifolia is included as an ingredient in a food product.

5. The method of claim 1, wherein the Morinda citrifolia is in capsule form.

6. The method of claim 1, wherein said dose of Morinda citrifolia inhibits the production of COX-2 related pro staglandins that cause pain and inflammation and inhibits to a lesser extent, the production of COX-1 related prostaglandins.

7. The method of claim 1, wherein said dose of Morinda citrifolia is administered at a predetermined concentration.

8. The method of claim 7, wherein said Morinda citrifolia is included as an ingredient in another food product.

9. A method of treating pain and inflammation comprising the steps of:
obtaining a quantity of Morinda citrifolia juice and pulp; filtering the wet pulp from the juice;
pasteurizing the juice; and
providing a dose of said Morinda citrifolia juice for consumption.

10. The method of claim 9, wherein the Morinda citrifolia juice is included as an ingredient in a food product.

11. The method of claim 9, wherein said dose is administered in an amount that is predetermined to limit undesired COX-1 inhibition relative to COX-2 inhibition.

12. A method of treating pain and inflammation comprising the steps of:
obtaining a quantity of Morinda citrifolia juice and pulp; filtering the wet pulp from the juice, wherein the wet pulp has a fiber content of from 10% to 40%, by weight;
pasteurizing the pulp; and
providing a therapeutic dose of said Morinda citrifolia pulp for consumption.

13. The method of claim 12, wherein the Morinda citrifolia pulp is included as an ingredient in a food product

14. The method of claim 12, wherein said dose is administered in an amount that is predetermined to limit undesired COX-1 inhibition relative to COX-2 inhibition.
